(19) United States
(12) Patent Application Publication
(10) Pub. No.: US 2003/0017567 A1
(43) Pub. Date: Jan. 23, 2003

(54) Biological sample processing methods and compositions that include surfactants

(75) Inventors: Ranjani V. Parthasarathy, Woodbury, MN (US); Raj Rajagopal, Woodbury, MN (US); William Bedingham, Woodbury, MN (US); Michael D. Dirksen, Stillwater, MN (US); Christopher J. Larson, Roseville, MN (US); Vinod P. Menon, Woodbury, MN (US)

Correspondence Address: Attention: Paul W. Busse, Office of Intellectual Property Counsel, 3M Innovative Properties Company, P.O. Box 33427, St. Paul, MN, 55133-3427, US

(73) Assignee: 3M Innovative Properties Company

(21) Appl. No.: 09/841,264

(22) Filed: Apr. 24, 2001

(51) Int. Cl. C12N 9/12

(52) U.S. Cl. 435/194

(57) ABSTRACT

Compositions containing an enzyme, a dye, and an effective amount of a surfactant selected from the group of a nonionic surfactant, a zwitterionic surfactant, and a mixture thereof and methods for processing sample materials.

### BACKGROUND

Many different chemical, biochemical, and other reactions include thermal cycling. Examples of thermal processes in the area of genetic amplification include, but are not limited to, Polymerase Chain Reaction (PCR), Sanger sequencing, etc. The reactions may be enhanced or inhibited based on the temperatures of the materials involved. Although it may be possible to process samples individually and obtain accurate sample-to-sample results, individual processing can be time-consuming and expensive.

One approach to reducing the time and cost of thermally processing multiple samples is to use a device including multiple chambers in which different portions of one sample or different samples can be processed simultaneously. When multiple reactions are performed in different chambers, however, one significant problem can be accurate control of chamber-to-chamber temperature uniformity. The need for accurate temperature control may manifest itself as the need to maintain a desired temperature in each of the chambers, or it may involve a change in temperature, e.g., raising or lowering the temperature in each of the chambers to a desired setpoint. In reactions involving a change in temperature, the speed or rate at which the temperature changes in each of the chambers may also pose a problem. For example, slow temperature transitions may be problematic if unwanted side reactions occur at intermediate temperatures. Alternatively, temperature transitions that are too rapid may cause other problems. As a result, another problem that may be encountered is comparable chamber-to-chamber temperature transition rate.

Another problem that may be encountered in those reactions in which thermal cycling is required is overall speed of the entire process. For example, multiple transitions between upper and lower temperatures may be required. Alternatively, a variety of transitions (upward and/or downward) between three or more desired temperatures may be required. In some reactions, e.g., polymerase chain reaction (PCR), thermal cycling must be repeated up to thirty or more times. Typical thermal cycling devices and methods that attempt to address the problems of chamber-to-chamber temperature uniformity and comparable chamber-to-chamber temperature transition rates, however, typically suffer from a lack of overall speed—resulting in extended processing times that ultimately raise the cost of the procedures.

One or more of the above problems may be implicated in a variety of chemical, biochemical and other processes. Examples of some reactions that may require accurate chamber-to-chamber temperature control, comparable temperature transition rates, and/or rapid transitions between temperatures include, e.g., the manipulation of nucleic acid samples to assist in the deciphering of the genetic code. See, e.g., J. Sambrook and D. W. Russell, Molecular Cloning, A Laboratory Manual 3^{rd} edition, Cold Spring Harbor Laboratory (2001). Nucleic acid manipulation techniques include amplification methods such as polymerase chain reaction (PCR); target polynucleotide amplification methods, such as self-sustained sequence replication (3SR); methods based on amplification of probe DNA, such as ligase chain reaction (LCR) and QB replicase amplification (QBR); transcription-based methods, such as ligation activated transcription (LAT) and nucleic acid sequence-based amplification (NASBA); and various other amplification methods, such as repair chain reaction (RCR) and cycling probe reaction (CPR).

One common example of a reaction in which all of the problems discussed above may be implicated is PCR amplification. Traditional thermal cycling equipment for conducting PCR uses polymeric microcuvettes that are individually inserted into bores in a metal block. The sample temperatures are then cycled between low and high temperatures, e.g., 55° C. and 95° C. for PCR processes. When using the traditional equipment according to the traditional methods, the high thermal mass of the thermal cycling equipment (which typically includes the metal block and a heated cover block) and the relatively low thermal conductivity of the polymeric materials used for the microcuvettes result in processes that can require two, three, or more hours to complete for a typical PCR amplification.

Another problem experienced in the preparation of finished samples (e.g., isolated or purified samples of, e.g., nucleic acid materials such as DNA, RNA, etc.) of human, animal, plant, or bacterial origin from raw sample materials (e.g., blood, tissue, etc.) is the number of thermal processing steps and other methods that must be performed to obtain the desired end product (e.g., purified nucleic acid materials). In some cases, a number of different thermal processes must be performed, in addition to filtering and other process steps, to obtain the desired finished samples.

One example is in the preparation of a finished sample (e.g., purified nucleic acid materials) from a starting sample (e.g., a raw sample such as blood, bacterial lysate, etc.). To obtain a purified sample of the desired materials in high concentrations, the starting sample must be prepared for, e.g., PCR, after which the PCR process is performed to obtain a desired PCR product. The PCR product is then subject to further manipulation such as sequencing, ligation, electrophoretic analysis, etc.

One method of improving conventional thermal cycling processes involves the use of electromagnetic radiation and energy absorbing pigments and dyes to absorb the radiation and convert it to thermal energy. The use of electromagnetic radiation absorbers such as pigments and dyes can interfere with reactions that involve the use of an enzyme. The enzyme can be deactivated, thereby preventing the formation of the desired products, e.g., PCR amplification products. Thus, there is a need for methods that allow for the use of electromagnetic radiation and absorbers such as dyes without adverse affects on the formation of the desired reaction products.

### SUMMARY OF THE INVENTION

The present invention provides various compositions and methods that involve the use of an enzyme and a dye, such as a near-infrared (near-IR or NIR) dye. Such compositions and methods are preferably used for processing sample mixtures that include biological materials. Preferred methods involve the use of thermal cycling of a sample material that includes a biological material and an enzyme through the application of electromagnetic energy. A dye is used to convert the electromagnetic energy into thermal energy and a surfactant is used to inhibit (i.e., reduce, prevent, and/or reverse) interaction between the enzyme and the dye. As used herein, inhibiting interaction between the enzyme and the dye involves reducing the interaction compared to the same system when the surfactant is not present. Preferably, inhibiting interaction between the enzyme and the dye involves preventing the interaction from occurring and/or substantially completely reversing such interaction.

The present invention provides a composition that includes an enzyme, a dye, and an effective amount of a surfactant selected from the group of a nonionic surfactant, a zwitterionic surfactant, and a mixture thereof, wherein the dye inactivates the enzyme in the absence of the surfactant, and the surfactant inhibits (i.e., reduces, prevents, and/or reverses) such inactivation. Preferably, the dye is selected from the group of a near-IR dye, a uv/visible dye, a fluorescent dye, and a mixture thereof. Preferably, the enzyme is a polymerase or a ligase.

A preferred composition of the present invention includes a polymerase enzyme, a near-IR dye (preferably, a diimminium dye or a cyanine dye), and an effective amount of a surfactant selected from the group of a nonionic surfactant, a zwitterionic surfactant, and a mixture thereof (preferably, a nonionic surfactant), wherein the near-IR dye inactivates the enzyme in the absence of the surfactant, and the surfactant inhibits the inactivation.

The present invention also provides a method of stabilizing an enzyme in a fluid sample in the presence of a dye under conditions that normally inactivate the enzyme. The method includes combining an effective amount of a surfactant selected from the group of a nonionic surfactant, a zwitterionic surfactant, and a mixture thereof, with the enzyme and the dye, wherein the surfactant inhibits inactivation of the enzyme. Preferably, the enzyme is a polymerase enzyme, the dye is a near-IR dye, and the surfactant is a nonionic surfactant.

The present invention also provides a method of conducting a thermal process that includes: providing a sample mixture that includes a biological material, an enzyme, an effective amount of a surfactant selected from the group of a nonionic surfactant, a zwitterionic surfactant, and a mixture thereof, and a dye at a first temperature (preferably, within a range of about 0° C. to about 50° C., and more preferably, about 20° C. to about 50° C.); and directly heating the sample mixture to a second temperature (preferably, within a range of about 50° C. to about 95° C.) higher than the first temperature; wherein the dye inactivates the enzyme in the absence of the surfactant and the surfactant inhibits the inactivation. Preferably, the method further includes cooling the sample mixture and directly reheating the sample mixture in a thermal cycling process, which preferably includes at least about 25 cycles, and preferably includes heating between a temperature of about 50° C. and about 95° C.

The present invention also provides a method of conducting a thermal cycling process that includes: providing a device that includes at least one process chamber that defines a volume for containing a sample mixture that includes a biological material, an enzyme (preferably, a polymerase enzyme), a dye (preferably, a near-IR dye), and a surfactant selected from the group of a nonionic surfactant, a zwitterionic surfactant, and a mixture thereof (preferably, a nonionic surfactant); delivering electromagnetic (i.e., nonthermal) energy to the process chamber to raise the temperature of the sample material in the process chamber; wherein the dye converts the electromagnetic energy into thermal energy; wherein the surfactant inhibits interaction between the enzyme and the dye. Preferably, the method further includes cooling the sample mixture and reheating the sample mixture in a thermal cycling process, which preferably includes at least about 25 cycles, and preferably includes heating between a temperature of about 50° C. and about 95° C.

Yet another method is one that involves denaturing hydrogen-bonded molecules. The method includes providing a sample mixture that includes hydrogen-bonded molecules, an enzyme, an effective amount of a surfactant selected from the group of a nonionic surfactant, a zwitterionic surfactant, and a mixture thereof, and a dye at a first temperature; and directly heating the sample mixture to a second temperature higher than the first temperature effective to denature the hydrogen-bonded molecules; wherein the dye inactivates the enzyme in the absence of the surfactant and the surfactant inhibits the inactivation.

As used in connection with the present invention, “thermal processing” (and variations thereof) means controlling (e.g., maintaining, raising, or lowering) the temperature of sample materials to obtain desired reactions. As one form of thermal processing, “thermal cycling” (and variations thereof) means sequentially changing the temperature of sample materials between two or more temperature setpoints to obtain desired reactions. Thermal cycling may involve, e.g., cycling between lower and upper temperatures, cycling between lower, upper, and at least one intermediate temperature, etc.

As used herein, “directly” heating a sample mixture means that the sample mixture is heated from within as opposed to heated upon transfer of thermal energy from an external source (e.g., heated container).

A composition containing a dye is “stable” in a thermal cycling process (due to the presence of a surfactant) if the dye displays no more than about a 20% decrease in absorbance relative to a control, i.e., the same composition not exposed to a thermal cycling process.

These and other features and advantages of the methods and compositions of the invention are described below with respect to illustrative and preferred embodiments of the invention.

### BRIEF DESCRIPTION OF THE DRAWINGS

FIG. 1 is a graph of temperature versus time of a laser cycling experiment using a NIR-dye and a surfactant.

### DETAILED DESCRIPTION OF PREFERRED EMBODIMENTS OF THE INVENTION

The present invention provides various compositions and methods that involve the use of a dye, such as a near-infrared (near-IR or NIR) dye, and a surfactant. Certain embodiments include the use of an enzyme in combination with a dye and a surfactant. Such compositions and methods are preferably used for processing sample mixtures (preferably, fluid samples) that include biological materials. Preferred embodiments of the present invention provide methods that involve thermal processing, e.g., sensitive chemical processes that involve the use of an enzyme such as a polymerase or a reverse transcriptase, such as PCR amplification, ligase chain reaction (LCR), self-sustaining sequence replication, enzyme kinetic studies, homogeneous ligand binding assays, and more complex biochemical or other processes that use an enzyme and require precise thermal control and/or rapid thermal variations. The methods involve the use of absorptive dyes, such as near-infrared dyes, that facilitate rapid and accurate electromagnetic energy-based or energy-derived thermal processing of sample materials.

Typically, absorptive dyes used to convert the electromagnetic energy into thermal energy are not inert in enzymatic reactions. Advantageously, a surfactant (or mixture of surfactants, preferably, a nonionic or zwitterionic surfactant, and more preferably, a nonionic surfactant, can be used to inhibit interaction between an enzyme (or mixture of enzymes) and a dye (or mixture of dyes). As used herein, inhibiting interaction between an enzyme and a dye involves reducing the interaction compared to the same system when the surfactant is not present. Preferably, inhibiting interaction between an enzyme and a dye involves preventing the interaction from occurring and/or substantially completely reversing such interaction.

Typically, absorptive dyes, particularly near-IR dyes, used to convert the electromagnetic energy into thermal energy are not stable in thermal processes (e.g., thermal cycling processes). Advantageously, a surfactant (or mixture of surfactants), preferably a nonionic or zwitterionic surfactant, and more preferably, a nonionic surfactant, can be used to stabilize a composition containing a near-IR dye in a thermal cycling process that includes cycling (preferably, at least 10 cycles, more preferably, at least 40 cycles) between about 50° C. and about 95° C. Preferably, the near-IR dye is a diimminium dye or a cyanine dye.

Typical methods of the present invention involve thermal processing of a sample mixture, such as a fluid (e.g., liquid) sample mixture, preferably one that includes a biological material. The methods generally include providing a sample mixture at a first temperature (preferably, within a range of about 0° C. to about 50° C., and more preferably, within a range of about 20° C. to about 50° C.), directly heating the sample mixture to a second temperature (preferably, within a range of about 50° C. to about 95° C.) higher than the first temperature. Preferably, the methods further include cooling the sample mixture and directly reheating the sample mixture in a thermal cycling process, which preferably includes at least about 25 cycles.

Conventional systems heat a sample mixture by heating the media surrounding the container (e.g., air, aluminum block, fluid). In contrast, the methods of the present invention provide one or more of the following advantages: noncontact heating where the fluid mixtures are heated directly without necessarily heating the surrounding container; ability to modify absorption coefficient to minimize thermal gradients, no need for support structures or heating elements (e.g., resistive heating elements, thermoelectric modules, etc.); and no need for good contact or registration with the heating block so the properties of the block (e.g., dimensions, material properties, conductivity, geometry) are not of significant importance. Also, because the “heating element” is in solution, localized heating can occur rapidly without necessarily heating the surrounding structure. In addition, because the surrounding structure is not heated, it facilitates removal of heat from the fluid compartment by diffusion. An added advantage is that one can control rate of heating by tailoring the concentration of near-IR absorber in solution (this in turn would allow one to adjust the amount of energy absorbed in the fluid compartment). In a similar fashion, the concentration of absorber can be adjusted to minimize thermal gradients occurring in the well. Because the wavelength maximum of the dye can be specified, a greater selection of light sources can be utilized (white light, uv light, near-IR laser) for noncontact heating the maximum absorption wavelength of the dye.

Recent publications have demonstrated prototype systems that use white light to heat the fluid directly, presumably using the near-IR content of light to heat water bands. In these systems, it is difficult to optimize heating (rapid, minimal thermal gradients) because of the inability to specify absorption coefficients.

Typical enzymes that can be adversely affected by large concentrations of absorptive dyes include, but are not limited to, polymerases, restriction endonucleases, and modifying enzymes (agarases, glycolysases, kinases, ligases, methylases, nucleases, proteases, phosphatases, reverse transciptases, topisomerases, and transferases). Preferably, the enzyme is a polymerase or a ligase, and more preferably, a polymerase, examples of which are well known to those of skill in the art.

Although many of the enzymes described above are adversely affected by large concentrations of absorptive dyes, some enzymes are not adversely affected. For such combinations in which the enzyme is not adversely affected by the dye, the use of a surfactant as described herein is not necessarily required to reduce, prevent, and/or reverse any adverse interaction between the dye and the enzyme; however, the surfactant may still provide advantage with respect to thermal degradation of the dye (discussed in greater detail below).

Suitable absorptive dyes that can be used to advantage in the methods of the present invention include those that will absorb energy, preferably, at a wavelength of at least about 400 nm, more preferably, at a wavelength of at least about 700 nm, and most preferably, at a wavelength of at least about 780 nm. Suitable absorptive dyes that can be used to advantage in the methods of the present invention include those that will absorb energy, preferably, at a wavelength of no greater than about 2000 nm, more preferably, at a wavelength of no greater than about 1300 nm, and most preferably, at a wavelength of no greater than about 1000 nm. These portions of the electromagnetic spectrum include the visible and near infrared (near-IR) portions. Other suitable absorptive dyes include fluorescent dyes. Preferably, the dye is a near-IR dye (e.g., a cyanine dye or a diimminium dye), an ultraviolet/visible (uv/vis) dye (e.g., dichlorophenol, indophenol, saffranin, crystal violet, and commercially available food coloring), a fluorescent dye (e.g., oligreen), or mixtures thereof. More preferably, the dye is a near-IR dye. The near-infrared (near-IR or NIR) region is typically about 700 nm to about 2000 nm, with the region of about 780 nm to about 1300 nm being of particular importance.

Classes of suitable absorptive dyes include acridine dyes, xanthene dyes, quinoneimine dyes, anthraquinone dyes, cartenoid dyes, nitro dyes, diazonium dyes, tetrazolium dyes, and di- and tri-aryl methane dyes. Classes of NIR dyes (i.e., NIR absorbers) include nitroso dyes, cyanine dyes, nigrosine dyes, triphenymethane dyes, imminium dyes, diimminium dyes, squarilium dyes, croconium dyes, nickel dithiolene dyes, quinone dyes, phthalocyanine dyes, azo dyes, indoaniline dyes, sulfur-containing dyes, vat dyes, and the like. Specific examples of suitable dyes include, but are not limited to, Methylene blue-thiazine dyes, Saffranin-Quinone imine dyes, Crystal Violet-triaryl methane dyes, Dichlorophenol indophenol-Quinone Imine dyes, as well as those listed in Table 1, etc. Preferably, the dye is a cyanine dye or a diimminium dye.

In the compositions and methods of the invention, a dye (or mixture of dyes) is used in an amount effective to heat the composition upon the application of electromagnetic energy. This amount will vary depending on the other components in the composition. Typically, such dyes can adversely affect an enzymatic reaction if used in an amount of 0.005 milligram/milliliter (mg/mL) of dye or greater. For effective heating, preferably, a dye is present in a reaction mixture at a concentration of at least about 0.1 mg/mL.

Preferably, the dyes are prepared as aqueous mixtures shortly before use. In some situations, aqueous solutions of dyes that are stored for 1 week and longer do not demonstrate benefit from the addition of a surfactant with respect to inactivation of an enzyme, although the surfactant may inhibit thermal degradation of the dye. However, when an aqueous solution of dye is added to a surfactant and stored for the same time period, it does benefit from the addition of a surfactant. Thus, for effective inhibition of inactivation of an enzyme by a dye, it is often desirable to use freshly prepared dye solutions.

TABLE 1

| DYE | CLASS | SOURCE |
|---|---|---|
| 785 WS | Cyanine | Spectra Colors, Kearny, NJ |
| 830 WS | Cyanine | Spectra Colors, Kearny, NJ |
| IR 768 | Cyanine | Aldrich, Milwaukee, WI |
| IR 780 | Cyanine | Aldrich, Milwaukee, WI |
| IR 792 | Cyanine | Aldrich, Milwaukee, WI |
| IR 1040 | Cyanine | Aldrich, Milwaukee, WI |
| | (thiapyrilium | |
| | compounds) | |
| IR 1100 | Cyanine | Aldrich, Milwaukee, WI |
| | (pyrilium compounds) | |
| Indocyanine green | Cyanine | Aldrich, Milwaukee, WI |
| SDA 8080 | Diimminium | H.W. Sands, Jupiter, FL |
| SDA 2141 | Cyanine | H.W. Sands, Jupiter, FL |
| SDA 8327 | Cyanine | H.W. Sands, Jupiter, FL |

Suitable surfactants that provide advantage in certain embodiments of compositions and methods of the present invention include those that will at least reduce interaction between a dye and an enzyme such that a desired product (e.g., PCR product) can be formed. Preferably, a surfactant substantially completely prevents such interaction and/or reverses such interaction. Other suitable surfactants that provide advantage in certain embodiments of compositions and methods of the present invention include those that will stabilize the dye. Preferred surfactants include nonionic and zwitterionic surfactants.

Examples of nonionic surfactants include, but are not limited to, esters of fatty acids and polyhydric alcohols, fatty acid alkanolamides, ethoxylated fatty acids, ethoxylated aliphatic acids, ethoxylated fatty alcohols, ethoxylated aliphatic alcohols, ethoxylated sorbitol fatty acid esters, ethoxylated glycerides, ethoxylated block copolymers with EDTA (ethylene diaminetetraacetic acid), ethoxylated cyclic ether adducts, ethoxylated amide and imidazoline adducts, ethoxylated amine adducts, ethoxylated mercaptan adducts, ethoxylated condensates with alkyl phenols, ethoxylated nitrogen-based hydrophobes, ethoxylated polyoxypropylenes, polymeric silicones, fluorinated surfactants, and polymerizable surfactants. Examples of fluorinated surfactants include those available under the trade names FLUORAD-FS 300 (Minnesota Mining and Manufacturing Co., St. Paul, Minn.) and ZONYL (Dupont de Nemours Co., Wilmington, Del.). Examples of polymerizable (reactive) surfactants include SAM 211 (alkylene polyalkoxy sulfate surfactant available under the trade name MAZON (PPG Industries, Inc., Pittsburgh, Pa.). Examples of zwitterionic surfactants include, but are not limited to, alkylamido betaines and amine oxides thereof, alkyl betaines and amine oxides thereof, sulfo betaines, hydroxy sulfo betaines, amphoglycinates, amphopropionates, balanced amphopolycarboxyglycinates, and alkyl polyaminoglycinates. Proteins have the ability of being charged or uncharged depending on the pH; thus, at the right pH, a protein, preferably with a pI of about 8 to 9, such as modified Bovine Serum Albumin or chymotrypsinogen, could function as a zwitterionic surfactant. Specific examples include those listed in Table 2 below. Various mixtures of surfactants can be used if desired.

A surfactant is used in an amount effective to produce the desired result (e.g., inhibit inactivation of an enzyme or stabilize a dye). Preferably, a surfactant is used in an amount of at least about 0.5 wt-%, more preferably, greater than about 0.5 wt-%, even more preferably, at least about 1 wt-%, and most preferably, greater than about 1 wt-%. Preferably, no more than about 20 wt-%, and more preferably, no more than about 4 wt-% of surfactant is needed to effectively reduce the interaction between the dye and the enzyme. Such percentages are typically based on a weight per volume of a sample mixture.

TABLE 2

| | | |
|---|---|---|
| SURFACTANT | | |
| TRADE NAME | SURFACTANT | SUPPLIER |

NONIONIC SURFACTANTS

| | | |
|---|---|---|
| PLURONIC F127 | Modified oxyethylated alcohol and/or | Sigma |
| | oxypropylated straight chain alcohols | St. Louis, |
| | | MO |
| TWEEN 20 | Polyoxyethylene (20) sorbitan | Sigma |
| | monolaurate | St. Louis, |
| | | MO |
| TRITON X-100 | Octyl phenoxy polyethoxyethanol | Sigma |
| | | St. Louis, |
| | | MO |
| BRIJ 97 | Polyoxyethylene (10) oleyl ether | Sigma |
| | | St. Louis, |
| | | MO |
| NONIDET P-40 | Nonyl phenoxy poly (ethyleneoxy) | Sigma |
| | ethanol | St. Louis, |
| | | MO |
| TOMADOL 1-7 | Ethoxylated alcohol | Tomah |
| | | Products |
| | | Milton, WI |
| Vitamin E TPGS | d-Alpha tocopheryl polyethylene glycol | Eastman |
| | 1000 | Kingsport, |
| | | TN |

ZWITTERIONIC SURFACTANTS

| | | |
|---|---|---|
| CHAPS | Cholamido propyl dimethyl ammonium | Sigma |
| | propanesulfonate | St. Louis, |
| | | MO |

Optionally, and preferably, radical scavengers can also be used to advantage in the compositions and methods of the present invention. Typically, continuous cycling causes a dye to be bleached (as a result of, for example, oxidization or degradation) such that it loses its color and hence, its efficiency in absorbing electromagnetic radiation. Although this could be used to advantage (as when the near-IR absorption is desirably destroyed resulting in the ability to interrogate a fluorescence signal from DNA/RNA labeled with near-IR tags), it is generally undesirable. It has been discovered that the use of a radical scavenger such as an antioxidant can inhibit this photodegradation (i.e., optical degradation) of the dye.

Suitable antioxidants include, but are not limited to, anoxomer, ascorbic acid, ascorbyl palmitate, ascorbyl stearate, butylated hydroxyanisole, butylated hydroxytoluene, t-butyl hydroquinone, calcium lactate, citric acid, clove extract, coffee bean extract, dilaurylthiodipropionate, disodium EDTA, dodecyl gallate, edetic acid, erythrobic acid, 6-ethoxy-1,2-dihydro-2,2,4-trimethylquinoline, eucalyptus extract, fumaric acid, gallic acid, gentian extract, guaiac gum, n-heptyl-p-hydroxybenzoate, heptyl paraben, hesperstin, 4-hydroxymethyl-2,6-di-t-butylphenol, isopropyl citrate, lecithin, nordihydroguairetic acid, octyl gallate, oryzanol, phosphatidyl choline, pimento extract, potassium metabisulfite, potassium sulfite, propylene glycol, rapeseed oil, rice bran extract, sage extract, sodium ascorbate, sodium sulfite, sodium tartarate, sodium thiosulfate, stannous chloride, sucrose, tocopherol, trihydroxybutyrophenene, as well as Vitamin A and derivatives thereof, Vitamin C and derivatives thereof, Vitamin E and derivatives thereof, phenols and hindered phenols, plant extracts, gallic acid and derivatives, quinines, and lecithins. Preferably, the addition of at least about 5 micrograms in a 25-microliter reaction (i.e., at least about 0.2 mg/mL) will protect the dye from bleaching.

In certain situations, the surfactant and the dye can be the same. For example, a dye molecule could be covalently attached to a hydrophilic chain like polyethylene glycol. This would yield a molecule with surfactant-like properties. Alternatively, the dye could be directly covalently attached to a surfactant molecule.

In certain situations, the surfactant and the antioxidant can be the same. For example, an antioxidant such as Viatmin E (hydrophobic) can be attached to a hydrophilic moiety such as polyethylene glycol. An example of such a compound would be Vitamine E-TPGS (tocopherol polyethylene glycol succinate).

Other agents that can be included in the compositions and methods of the present invention include a buffer, a reference dye, and other PCR reactants, which are discussed in greater detail below. Suitable buffers typically have a pH of about 4 to about 9, such as tris-HCl at a pH of between 8 and 9. Suitable reference dyes include ROX (carboxyrhodamine), TAMRA (carboxytetramethylrhodamine), FAM (carboxy fluorescien), and Texas Red, which are fluorescent dyes. Such reference dyes are typically used at lower concentrations than the dyes used for heating purposes (e.g., the NIR dyes discussed above).

Although the methods can be used in a variety of devices, a variety of illustrative embodiments of preferred devices are described in U.S. patent application Ser. No. 60/214,508 filed on Jun. 28, 2000 and entitled THERMAL PROCESSING DEVICES AND METHODS. Other useable device constructions may be found in, e.g., U.S. patent application Ser. No. 09/710,184 filed on Nov. 10, 2000 and entitled CENTRIFUGAL FILLING OF SAMPLE PROCESSING DEVICES.

Regardless of the specific device, a fluid sample (e.g., solution) in a process chamber can be interrogated by electromagnetic energy of selected wavelengths (if desired). Suitable electromagnetic energy is supplied by an electromagnetic energy source that directly heats the fluid (e.g., solution) in the process chamber source and is preferably remote from the device, i.e., it is not located on the device. Examples of some suitable electromagnetic energy sources may include, but are not limited to, lasers, broadband electromagnetic energy sources (e.g., white light), etc. The electromagnetic energy source may be provided continuously or intermittently based on a variety of factors, e.g., the desired temperature of the sample materials, the rate at which thermal energy is removed from each process chamber, the desired rate of temperature change, whether the process chambers include a reflective component, etc. If the electromagnetic energy source is cycled or otherwise varied, a registration system may be used to deliver a selected amount of energy to selected process chambers and an optional additional temperature control mechanism in the form of a fluid source, e.g., pressurized air or any other suitable fluid, can be directed at the surface of the device. These are discussed in U.S. patent application Ser. No. 60/214,508 filed on Jun. 28, 2000 and entitled THERMAL PROCESSING DEVICES AND METHODS.

The advantage of using an electromagnetic energy receptive material such as an electromagnetic absorbing dye (e.g., near infrared, visible dye) is that the sample materials in the device can be heated in the absence of physical contact with the device and without directly heating the container. For example, if the electromagnetic energy receptive material is sensitive to radio-frequency (RF) radiation, the device can be rotated such that the process chambers are resident within an RF field for sufficient time to obtain the desired heating. Similar noncontact heating may be obtained with microwave radiation, etc. It will, however, be understood that the form in which the electromagnetic radiation is provided should be compatible with the sample materials and desired reactions located within the process chambers.

The methods described herein can be used in a variety of different processes requiring thermal cycling of samples contained in the process chambers of the devices. Examples of some such processes involve chemical reactions of samples, e.g., nucleic acid amplification. For example, samples may be mixed with a polynucleotide, a polymerase (such as Taq polymerase), nucleoside triphosphates, a first primer hybridizable with the sample polynucleotide, and a second primer hybridizable with a sequence complementary to the polynucleotide. Some or all of the required reagents may be present in the device as manufactured, they may be loaded into the process chambers after manufacture of the device, they may be loaded in the process chambers just before introduction of the sample, or they may be mixed with sample before loading into the process chambers.

Although polynucleotide amplification by PCR is described in the most detail herein, the devices and methods of using them may be used for a variety of other systems that involve the use of dyes (and preferably, enzymes) in thermal processes, particularly those involving polynucleotide amplification reactions, ligand-binding assays, or denaturing hydrogen-bonded molecules.

Preferred reactions may be thermally cycled between alternating upper and lower temperatures, such as PCR, or they may be carried out at a single temperature, e.g., nucleic acid sequence-based amplification (NASBA). The reactions can use a variety of amplification reagents and enzymes, including DNA ligases, RNA polymerases and/or reverse transcriptases, etc. Polynucleotide amplification reactions that may be performed using the methods of the invention include, but are not limited to a) target polynucleotide amplification methods such as self-sustained sequence replication (3SR) and strand-displacement amplification (SDA); b) methods based on amplification of a signal attached to the target polynucleotide, such as “branched chain” DNA amplification; c) methods based on amplification of probe DNA, such as ligase chain reaction (LCR) and QB replicase amplification (QBR); d) transcription-based methods, such as ligation activated transcription (LAT) and nucleic acid sequence-based amplification (NASBA); and e) various other amplification methods, such as repair chain reaction (RCR) and cycling probe reaction (CPR).

Of the potential uses for the devices and methods of the present invention, PCR is one important such use, although it should be understood that the present invention is not limited to PCR amplification. PCR allows for analysis of extremely small amounts of target nucleic acid (e.g., DNA) using an excess of two oligonucleotide primers that are capable of flanking the region of the denatured molecule to be amplified and extending the nucleic acid molecule by nucleotide addition from the primers by the action of a polymerase enzyme (such as Taq DNA polymerase) in the presence of free deoxynucleoside triphosphates (dNTPs), resulting in a double replication of the starting target nucleic acid molecule. The nucleic acid molecules are again thermally treated to denature, and the process is repeated to form PCR amplification products (also referred to as PCR amplicons).

In some embodiments, DNA primers and probes are provided in the process chambers during manufacturing of specific devices. A DNA target sample could then be introduced into the process chambers to conduct PCR amplification of the DNA target. The target sample may include, e.g., target DNA, buffer, and polymerase enzyme.

After the target sample has been distributed to the process chambers (containing the pre-loaded primers and probes), the temperature of the materials in each of the process chambers can be raised to a selected base temperature (e.g., 60° C.) to begin the PCR amplification. A laser or other electromagnetic energy source can be used to raise the temperature of the sample materials in each of the process chambers to an upper target temperature at which, e.g., denaturing of the DNA occurs.

After reaching the target temperature, the sample materials are brought back down to the base temperature. This can occur by a variety of techniques. In one method of the present invention, the base temperature can be reached through convective cooling as the device rotates. That convective cooling alone, or in connection with conductive cooling using a base plate, impinging fluid jets, etc., preferably provides for rapid cooling of the sample materials, followed by rapid heating back up to the target temperature. The rapid heating and cooling is advantageous in that a desired number of thermal cycles can be completed in a relatively short period of time. Preferably, the methods described herein, whether for PCR or other techniques, a thermal cycling process preferably includes at least about 25 cycles, and preferably includes heating between a temperature of about 50° C. and about 95° C.

A preferred method involves the use of a device with a plurality of process chamber arrays such as those illustrated in U.S. patent application Ser. No. 60/214,508 filed on Jun. 28, 2000 and entitled THERMAL PROCESSING DEVICES AND METHODS. Each of the process chamber arrays include a number of chambers that are preferably arranged generally radially on a device (such that centrifugal forces can move fluids sequentially from chamber to chamber). The chambers within each of the arrays are in fluid communication using channels or other conduits that may, in some embodiments, include valve structures to control the movement as desired.

Using such a device, starting sample material, e.g., lysed blood cells, is provided in a loading chamber. A filter is preferably provided to filter the starting sample material as it moves from the loading chamber to a first process chambers. The first process chambers preferably include suitable PCR primers as supplied, e.g., dried down in each of the chambers. Each of the chambers may include the same primer or different primers depending on the nature of the investigation being performed on the starting sample material. One alternative to providing the primers in the process chambers before loading the sample is to add a suitable primer to the loading chamber with the starting sample material (provided that the primer is capable of passing through the filter, if present).

After locating the starting sample material and any required primers in the process chambers, the materials in the process chambers are thermally cycled under conditions suitable for PCR amplification of the selected genetic material.

After completion of the PCR amplification process, the materials in each of the first process chambers may be moved through another filter chamber (one filter chamber for each process chamber) to remove unwanted materials from the amplified materials, e.g., PCR primers, unwanted materials in the starting sample that were not removed by filter, etc. The filter chambers may, for example, contain size exclusion substances, such as permeation gels, beads, etc. (e.g., MicroSpin or Sephadex available from Amersham Pharmacia Biotech AB, Uppsala, Sweden).

After clean-up of the sample materials in the filter chambers, the filtered PCR amplification products from each of the first process chambers are moved into a pair of multiplexed second process chambers for, e.g., Sanger sequencing of the genetic materials amplified in the first process chambers through appropriate control of the thermal conditions encountered in second process chambers.

### EXAMPLES

Objects and advantages of this invention are further illustrated by the following examples, but the particular materials and amounts thereof recited in these examples, as well as other conditions and details, should not be construed to unduly limit this invention. All dye solutions used herein were freshly prepared (i.e., used within one day).

### Example 1: Preparation of a Mixture Containing a NIR Dye and a Surfactant

Typically, a fresh stock solution of 5 milligrams (mg) of a water-insoluble near-infrared dye (NIR dye) was dissolved in 50 microliters (μL) of dimethylsulfoxide (DMSO) and brought up in water to a final volume of 1 milliliter (mL). This was then diluted out to various concentrations of dye. The diluted dye (typically 0.2 mg/mL) was then added to a surfactant solution (typically 4 wt-%) to form a complex. The complex was added to a PCR reaction. The dyes and surfactants that were tested are listed in Tables 1 and 2, respectively.

### Example 2: Formation of PCR Product in the Presence of a Surfactant

PCR reactions were set up for a conserved sequence (327 bp) in the protease region of the genome of porcine endogenous retrovirus using a plasmid containing the PERV protease fragment, a forward primer and a reverse primer designed using a primer express program from Applied Biosystems, and AMPLITAQ DNA polymerase cocktail (commercially available from Applied Biosystems, Foster City, Calif.) and cycled in a thermocycler (9700, Applied Biosystems). The PCR reactions were typically carried out in 25-μL volumes. The NIR dye/surfactant mixture was added to the PCR cocktail to give the desired concentration of dye and surfactant in a total volume of 25 μL. A typical PCR cocktail contains 10x buffer, 2.5 μL; dNTPs, 2.0 μL (200 μM of each); forward primer, 0.5 μL (200 nM); reverse primer, 0.5 μL (200 nM); template DNA, 1.0 (1 ng/μL); Amplitaq, 0.25 μL (1.25 units); sterile water, 18.25 μL.

The products of PCR reactions were run on 1% agarose gel containing ethidium bromide and visualized using an ALPHA IMAGER (Alpha Innotech Corp., San Leandro, Calif.). In initial experiments, for example, it was found that for the dye 785 WS a final dye concentration of 0.005 mg/mL or higher inhibits PCR. However, a higher concentration of dye (0.1 mg/mL) for effective heating was needed. Thus, for all further PCR experiments, a dye concentration of 0.1 mg/mL or higher was used. The results of PCR reaction with dye/surfactant mixture showed that the dye alone inhibits PCR, while addition of surfactants restored the activity of PCR. Taq polymerase was tested from different manufacturers (Promega Corp., Madison, Wis.; Roche Biochemicals, Indianapolis, Ind.; Eppendorf, Scientific Inc., Westbury, N.Y.; Sigma, St. Louis, Mo.) and all the Taq polymerases were inhibited by 785 WS and SDA 8080. The addition of surfactants (PLURONIC or TWEEN ) restored the activity of the enzyme.

Quantitative PCR using TaqMan probes or Sybr green results for SDA 8080 (0.2 mg/mL) and 785 WS (0.1 mg/mL) indicated that the dye alone inhibited the assay, while the addition of surfactants, PLURONIC F127 or TWEEN-20, restored the activity of the assay in both cases. This experiment indicated that the dye/surfactant mixture did not interfere with the fluorescence measurements of the TaqMan probes and the exonuclease activity of the Taq polymerase in addition to the polymerase activity.

In order to find out the optimum concentration of surfactant for PCR with NIR dye (785 WS and SDA 8080), the surfactant concentration in PCR reaction was varied from 0.1 wt-% to 4 wt-%. The PCR product formed was quantitated using DNA 7500 Labchip kit in Agilent 2100 Bioanalyzer (Agilent Inc, Paloalto, Calif.). The results shown in Table 3 indicate that increasing the concentration of surfactant relative to dye assists in the formation of PCR product.

TABLE 3

| | | 785 WS dye | PCR | SDA 8080 dye | PCR |
|---|---|---|---|---|---|
| | | concentration | Product | concentration | Product |
| | Wt % | (mg/mL) | (ng/μL) | (mg/mL) | (ng/μL) |
| Control | 0 | 0 | 17 | 0 | 25 |
| PLURONIC | 4 | 0 | 32* | 0 | 32* |
| F-127 | 0 | 0.1 | 0 | 0.2 | 0 |
| | 0.1 | 0.1 | 0 | 0.2 | 0 |
| | 0.5 | 0.1 | 0.1 | 0.2 | 0 |
| | 1 | 0.1 | 4.7 | 0.2 | 0 |
| | 2 | 0.1 | 12.5 | 0.2 | 0.7 |
| | 4 | 0.1 | 15 | 0.2 | 10 |
| Control | 0 | 0 | 20.8 | 0 | 27 |
| TWEEN-20 | 4 | 0 | 28* | 0 | 28* |
| | 0 | 0.1 | 0 | 0.2 | 0 |
| | 0.1 | 0.1 | 0 | 0.2 | 27 |
| | 0.5 | 0.1 | 12.7 | 0.2 | 29 |
| | 1 | 0.1 | 19 | 0.2 | 31 |
| | 2 | 0.1 | 23.7 | 0.2 | 22 |
| | 4 | 0.1 | 24 | 0.2 | 19 |
| Control | 0 | 0 | 11.2 | 0 | 8.4 |
| TRITON | 4 | 0 | 23.8* | 0 | 23.8* |
| X-100 | 0 | 0.1 | 0 | 0.2 | 0 |
| | 0.1 | 0.1 | 0 | 0.2 | 0 |
| | 0.5 | 0.1 | 0.1 | 0.2 | 2.3 |
| | 1 | 0.1 | 0.4 | 0.2 | 1.4 |
| | 2 | 0.1 | 2.2 | 0.2 | 1.9 |
| | 4 | 0.1 | 1.3 | 0.2 | 1.1 |
| Control | 0 | 0 | 27 | 0 | 28 |
| NP-40 | 4 | 0 | 24.2* | 0 | 24.2* |
| | 0 | 0.1 | 0 | 0.2 | 0 |
| | 0.1 | 0.1 | 0 | 0.1 | 0 |
| | 0.5 | 0.1 | 0 | 0.2 | 23.3 |
| | 1 | 0.1 | 0 | 0.2 | 23.7 |
| | 2 | 0.1 | 0.3 | 0.2 | 17.2 |
| | 4 | 0.1 | 3.8 | 0.2 | 18 |
| Control | 0 | 0 | 29 | 0 | 29 |
| BRIJ 97 | 4 | 0 | Nd | 0 | nd |
| | 0 | 0.1 | 0 | 0.2 | 0 |
| | 0.1 | 0.1 | 0 | 0.2 | 18.7 |
| | 0.5 | 0.1 | 0 | 0.2 | 19.3 |
| | 1 | 0.1 | 0 | 0.2 | 23.5 |
| | 2 | 0.1 | 13.8 | 0.2 | 25.5 |
| | 4 | 0.1 | 21.9 | 0.2 | 22.6 |

*denotes data obtained from a different experiment under identical PCR conditions all data averaged from experiments performed in triplicate
nd = not determined

### Example 3. Interaction Between an Enzyme and a Dye Is Reversible Using a Surfactant

In order to understand the reason for inhibition of PCR by a NIR dye, two separate experiments were carried out. In a first experiment (Example 3A), Taq polymerase was allowed to interact with 785 WS dye (40 micrograms (μg)) at room temperature for 25 minutes. In a second experiment (Example 3B), template DNA was allowed to interact with the same dye under the same conditions. In both experiments, the dye was removed from the enzyme or DNA template by passing the solution through a CENTRISEP column (Princeton Separations, Adelphia, N.J.). The Taq polymerase or template DNA was then used in separate reactions (25 μL) for amplifying PERV protease fragment as described in Example 2. The final dye concentration in the PCR cocktail was at 1.6 mg/mL. The results of the PCR process in Example 3A showed that Taq polymerase was inactivated by exposure to the dye thereby resulting in no amplification. From a Example 3B, the results of the PCR process showed that the dye did not have any effect on the DNA template as the removal of the dye from the template resulted in amplification of the expected fragment. From the above experiments, it was clear that the enzyme was being inhibited by the dye. In a separate experiment, Taq polymerase was allowed to react with a NIR dye (0.2 mg/mL for 785 WS and 0.3 mg/mL for SDA 8080) for 15 minutes at room temperature and the enzyme/dye mixture was added to the PCR cocktail containing different surfactants (4 wt-%). The reaction was set up to amplify PERV protease fragment as described in Example 2 and the products of the reaction were analyzed by agarose gel electrophoresis. The results of the PCR process showed that the effect of the dye on the enzyme was reversible by the addition of surfactants. The amount of the PCR product made was comparable to the controls.

### Example 4: RNA Polymerase Activity Is Restored by Addition of a Surfactant

RNA polymerase reactions were set up using T7 or SP6 RNA polymerase (Epicentre Technologies, Madison, Wis.) using a linearized plasmid template containing the T7 or SP6 promoter. The reagents were used according to manufacturer's instructions. The reaction products were on 1% agarose gel and the results indicated that the NIR dye 785 WS (0.1 mg/mL) inhibited activity of both the enzymes, while addition of surfactants (PLURONIC 4 wt-%) restored the enzyme activity. The dye SDA 8080 did not inhibit the activity of both enzymes at 0.2 mg/mL.

### Example 5: DNA Ligase Activity Is Restored by Addition of a Surfactant

DNA ligation reactions were set up using T4 DNA ligase (Life Technologies, Inc, Gaithesburg, Md.) using a linearized plasmid template (pET21a, Novagen, Madison, Wis.) and a DNA insert. The reagents were used according to manufacturer's instructions. Escherichia coli DH10B competent cells were transformed with the ligated products and transformed cells were plated on Clondisc (Clontech, Palo Alto, Calif.) with appropriate antibiotic (ampicillin at 50 μg/mL). After incubation of plates at 37° C. for 16 hours to 18 hours, the resulting colonies were counted and analysed for plasmid by miniprep and restriction digest. The NIR dyes 785 WS (0.1 mg/mL) and SDA 8080 (0.2 mg/mL) inhibited ligase activity as no colonies could be detected on the plates plated with the ligation mix containing the dye. The addition of surfactants (4 wt-% PLURONIC for 785 WS and 4 wt-% TWEEN-20 for SDA 8080) restored the activity of ligase as similar number of colonies were observed in control ligation mix (with or without surfactants) and the ligation mix containing dye/surfactant combination. In addition, all of the plasmids isolated from these plates contained the same insert as verified by restriction digest and DNA sequencing.

In addition to transformation and plating with the ligation mix, the ligated products were used as DNA template in a PCR reaction containing primers flanking the insert DNA on the plasmid template (T7 promoter and T7 terminator primers, Novagen). After PCR, the reaction products were run on 1% agarose gel and the results indicated the presence of expected DNA fragment from control ligation mix and ligation mix containing dye/surfactant combination but not from mix containing dye alone (785 WS or SDA 8080).

### Example 6: A Variety of Dyes Benefit by the Addition of a Surfactant

A variety of dyes (NIR dyes such as IR782, IR768, SDA 2141, SDA8327, IR 830 WS, fluorescent dyes like oligreen and visible dyes like dichlorophenol, indophenol, saffranin, crystal violet, and commercially available food coloring) at typical concentrations of 0.1 or 0.2 mg/mL were allowed to interact with 4 wt-% PLURONIC F127 and each mixture was added to the PCR cocktail. The reaction was set up to amplify PERV protease fragment as described in Example 2 and the products of the reaction were analyzed by agarose gel electrophoresis. The results of the PCR process showed that each of these dyes inhibited PCR, while addition of surfactants like PLURONIC F127 inhibited the effect of the dye on the reaction.

### Example 7: A Surfactant Stabilizes a Dye and Assists in Preventing Thermal Degradation

The dyes SDA 8080 and 785 WS were mixed with a variety of surfactants such as PLURONIC F-127, TWEEN-20, TRITON X-100, NP-40, BRIJ-97 and cycled in a thermocycler (9700, Applied Biosystems). Absorbance readings were taken at the beginning and end of thermal cycling using HP Chem Station (HP 8453, Agilent Technologies, Palo Alto, Calif.) at 785 nm for the 785 WS dye and 1040 nm for the SDA 8080 dye. The results indicated that the dye without the surfactant thermally degraded to levels that did not provide effective heating while the dye/surfactant mixture underwent significantly less degradation. Thus, the surfactant helps to protect thermal degradation of the dye. The data is shown in Table 4.

TABLE 4

| | | 785 WS (0.1 mg/mL) | | | SDA 8080 (0.2 mg/mL) | | |
|---|---|---|---|---|---|---|---|
| | | PCR | Thermal | Laser | PCR | Thermal | Laser |
| | wt % | Product | Degradation | Stability | Product | Degradation | Stability |
| PLURONIC | 0 | − | 33.7% | −* | − | 92.0% | + |
| F-127 | 0.1 | − | 29.9% | Nd | − | 90.5% | nd |
| | 0.5 | + | 2.3% | Nd | − | 73.8% | nd |
| | 1 | + | 4.8% | Nd | − | 46.0% | nd |
| | 2 | + | 4.6% | Nd | + | 28.9% | nd |
| | 4 | + | 0.5% | −* | + | 22.2% | + |
| | 12 | + | nd | Nd | + | nd | nd |
| TWEEN-20 | 0 | − | 33.7% | −* | − | 92.0% | + |
| | 0.1 | + | 2.9% | Nd | + | 87.6% | nd |
| | 0.5 | + | 2.3% | Nd | + | 37.5% | nd |
| | 1 | + | 6.0% | Nd | + | 14.5% | nd |
| | 2 | + | 3.8% | Nd | + | 10.1% | nd |
| | 4 | + | 5.4% | Nd | + | 5.6% | nd |
| | 20 | + | nd | Nd | + | 4.3% | + |
| TRITON | 0 | − | 35.0% | −* | − | 95.0% | + |
| X-100 | 0.1 | − | 24.8% | Nd | − | 76.0% | nd |
| | 0.5 | + | nd | Nd | + | nd | nd |
| | 1 | + | nd | Nd | + | nd | nd |
| | 2 | + | nd | Nd | + | nd | nd |
| | 4 | + | 4.6% | Nd | + | 0.0% | nd |
| | 20 | + | nd | −* | + | 7.1% | + |
| NP-40 | 0 | − | 35.0% | Nd | − | 95.0% | + |
| | 0.1 | − | 20.2% | Nd | − | 82.1% | nd |
| | 0.5 | − | nd | Nd | + | nd | nd |
| | 1 | − | nd | Nd | + | nd | nd |
| | 2 | + | nd | Nd | + | nd | nd |
| | 4 | + | 27.4% | Nd | + | 48.8% | nd |
| Vitamin E | 0 | − | 35.0% | −* | − | 100.0% | + |
| TPGS | 0.1 | − | nd | Nd | + | nd | nd |
| | 0.5 | + | 6.5% | Nd | + | 89.5% | nd |
| | 1 | + | nd | Nd | + | nd | nd |
| | 2 | + | 4.0% | Nd | + | 88.9% | nd |
| | 4 | + | 5.3% | + | + | 92.3% | nd |
| BRIJ 97 | 0 | − | 30.0% | −* | − | 95.7% | + |
| | 0.01 | − | 79.0% | Nd | + | 86.2% | nd |
| | 0.5 | + | 95.0% | Nd | + | 67.7% | nd |
| | 1 | + | 94.5% | Nd | + | 50.0% | nd |
| | 2 | + | 94.5% | Nd | + | 29.4% | nd |
| | 4 | + | 96.0% | Nd | + | 6.0% | nd |
| | 20 | − | nd | −* | − | nd | + |

Laser stability is a measure of the absorbance drop in the dye solution upon being exposed to a laser source.
A mark of “+” means that there is less than a 20% degradation in dye absorbance over a period of 25 exposures.
A mark of “−” means that there is a greater than 20% degradation in dye absorbance over a period of 25 exposures.
*can be made laser stable after addition of antioxidant

### Example 8: A NIR Dye Heats Water Efficiently with a Surfactant

This experiment demonstrates that the dye/surfactant mixture can heat very efficiently at concentrations that are PCR compatible. An 813-nm diode laser source at 1.2W (1.9A) (Opto Power Corp., Tucson, Ariz. ) was used to heat a 10μL volume of near-IR dye at a concentration (0.1 mg/mL for 785 WS, 0.2 mg/iL for SDA 8080) to yield a 35° C. rise in temperature. The laser was manually turned on long enough for the temperature in a 10-μL well to increase by 35° C. (1-3 seconds) and was manually turned off when that level was attained. The temperature data was obtained using a thermocouple in the well, and the transmittance data was obtained via a power meter whose signals were digitized and graphically interpreted.

PCR thermal cycling typically requires rapid increase in temperature from 60° C. to 95° C. In another series of experiments, it was demonstrated that a 35° C. increase in temperature from 60° C. to 94° C. was obtained in under 5 seconds. This demonstrates that the thermal requirements for PCR cycling can be met at dye and surfactant concentrations that would allow DNA amplification.

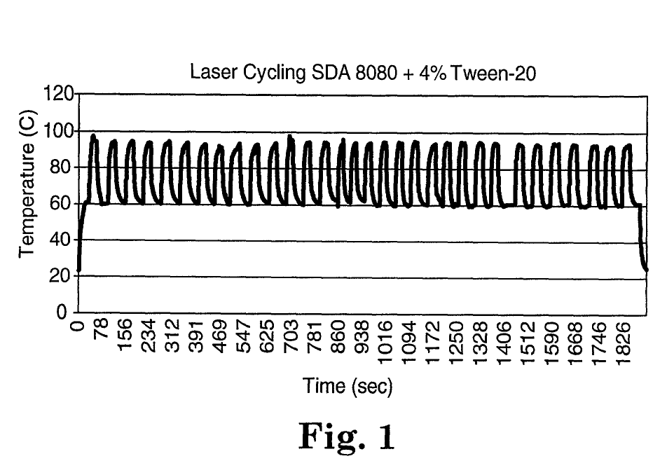

In another experiment, the 10 μL well was used to see if the dye/surfactant mixture could withstand laser cycling over a period of 30 cycles. The laser power was systematically varied from 60° C. to 94° C., 30 times. FIG. 1 shows SDA 8080 being cycled repeatedly.

The laser power did not change much during the course of cycling. This indicated that the SDA 8080 dye did not bleach to any significant extent over a period of 30 cycles.

### Example 9: An Antioxidant Reduces Photobleaching of a NIR Dye

Unlike in Example 8, some dyes and dye/surfactant complexes undergo photodegradation when exposed to laser beam. Incorporation of PCR-compatible radical scavengers like ascorbic acid may significantly reduce photodegradation for some of these dyes. Addition of ascorbic acid to virtually any of the surfactants tested with 785 WS reduced photobleaching significantly. Specifically, in the case of 785 WS/BRIJ-97, the addition of ascorbic acid (typically 5 mg/mL) helped reduce the amount of photobleaching from 100% to 20% after 30 cycles. In some circumstances, rapid/controlled photodegradation may be desired. In the case of SDA 8080, addition of ascorbic acid destroyed the absorbance in the visible and near-IR region of the dye. This would allow DNA tags (fluorescent or vis/IR) to be monitored at the end of the PCR reaction.

Vitamin E-TPGS has both surfactant as well as antioxidant properties. Thus, addition of Vitamin E-TPGS (4 wt-%) to 785 WS (0.1 mg/mL) showed that the antioxidant properties became apparent as light stability was significantly increased after 30 cycles of laser exposure.

Patents, patent applications, and publications disclosed herein are hereby incorporated by reference (in their entirety) as if individually incorporated. It is to be understood that the above description is intended to be illustrative, and not restrictive. Various modifications and alterations of this invention will become apparent to those skilled in the art from the foregoing description without departing from the scope of this invention, and it should be understood that this invention is not to be unduly limited to the illustrative embodiments set forth herein.

What is claimed is:

1. A composition comprising an enzyme, a dye, and an effective amount of a surfactant selected from the group of a nonionic surfactant, a zwitterionic surfactant, and a mixture thereof, wherein the dye inactivates the enzyme in the absence of the surfactant, and the surfactant inhibits such inactivation.

2. The composition of claim 1 wherein the dye is selected from the group of a near-IR dye, a uv/visible dye, a fluorescent dye, and a mixture thereof.

3. The composition of claim 2 wherein the dye is a near-IR dye.

4. The composition of claim 3 wherein the near-IR dye is a diimminium dye or a cyanine dye.

5. The composition of claim 1 wherein the enzyme is a polymerase or a ligase.

6. The composition of claim 1 wherein the nonionic surfactant is selected from the group of esters of fatty acids and polyhydric alcohols, fatty acid alkanolamides, ethoxylated fatty acids, ethoxylated aliphatic acids, ethoxylated fatty alcohols, ethoxylated aliphatic alcohols, ethoxylated sorbitol fatty acid esters, ethoxylated glycerides, ethoxylated block copolymers with EDTA, ethoxylated cyclic ether adducts, ethoxylated amide and imidazoline adducts, ethoxylated amine adducts, ethoxylated mercaptan adducts, ethoxylated condensates with alkyl phenols, ethoxylated nitrogen-based hydrophobes, ethoxylated polyoxypropylenes, polymeric silicones, fluorinated surfactants, polymerizable surfactants, and mixtures thereof.

7. The composition of claim 1 wherein the zwitterionic surfactant is selected from the group of alkylamido betaines and amine oxides thereof, alkyl betaines and amine oxides thereof, sulfo betaines, hydroxy sulfo betaines, amphoglycinates, amphopropionates, balanced amphopolycarboxyglycinates, and alkyl polyaminoglycinates, and mixtures thereof.

8. The composition of claim 1 wherein the dye is present at a concentration of at least about 0.005 mg/mL.

9. The composition of claim 1 wherein the effective amount of surfactant is at least about 0.5 wt-%.

10. The composition of claim 9 wherein the effective amount of surfactant is no greater than about 20 wt-%.

11. The composition of claim 1 further comprising a buffer.

12. The composition of claim 1 further comprising a dinucleotide triphosphate.

13. The composition of claim 1 further comprising a reference dye.

14. The composition of claim 1 further comprising an antioxidant.

15. The composition of claim 14 wherein the dye is capable of optical degradation.

16. The composition of claim 1 wherein the surfactant is an antioxidant.

17. A composition comprising a polymerase enzyme, a near-IR dye, and an effective amount of a surfactant selected from the group of a nonionic surfactant, a zwitterionic surfactant, and a mixture thereof, wherein the near-IR dye inactivates the enzyme in the absence of the surfactant, and the surfactant inhibits the inactivation.

18. A composition comprising:
a polymerase enzyme;
a near-IR dye selected from the group of a diimminium dye, a cyanine dye, and a mixture thereof, and
an effective amount of a nonionic surfactant;
wherein the near-IR dye inactivates the enzyme in the absence of the surfactant, and the surfactant inhibits the inactivation.

19. A composition comprising:
a polymerase enzyme;
a near-IR dye selected from the group of a diimminium dye, a cyanine dye, and a mixture thereof; and
an effective amount of a nonionic surfactant selected from the group of esters of fatty acids and polyhydric alcohols, fatty acid alkanolamides, ethoxylated fatty acids, ethoxylated aliphatic acids, ethoxylated fatty alcohols, ethoxylated aliphatic alcohols, ethoxylated sorbitol fatty acid esters, ethoxylated glycerides, ethoxylated block copolymers with EDTA, ethoxylated cyclic ether adducts, ethoxylated amide and imidazoline adducts, ethoxylated amine adducts, ethoxylated mercaptan adducts, ethoxylated condensates with alkyl phenols, ethoxylated nitrogen-based hydrophobes, ethoxylated polyoxypropylenes, polymeric silicones, fluorinated surfactants, polymerizable surfactants, and mixtures thereof;
wherein the near-IR dye inactivates the enzyme in the absence of the surfactant, and the surfactant inhibits the inactivation.

20. A method of stabilizing an enzyme in a fluid sample in the presence of a dye under conditions that normally inactivate the enzyme, the method comprising combining an effective amount of a surfactant selected from the group of a nonionic surfactant, a zwitterionic surfactant, and a mixture thereof, with the enzyme and the dye, wherein the surfactant inhibits inactivation of the enzyme.

21. The method of claim 20 wherein the dye is selected from the group of a near-IR dye, a uv/visible dye, a fluorescent dye, and a mixture thereof.

22. The method of claim 21 wherein the enzyme is a polymerase or a ligase.

23. The method of claim 20 wherein the surfactant is a nonionic surfactant selected from the group of esters of fatty acids and polyhydric alcohols, fatty acid alkanolamides, ethoxylated fatty acids, ethoxylated aliphatic acids, ethoxylated fatty alcohols, ethoxylated aliphatic alcohols, ethoxylated sorbitol fatty acid esters, ethoxylated glycerides, ethoxylated block copolymers with EDTA, ethoxylated cyclic ether adducts, ethoxylated amide and imidazoline adducts, ethoxylated amine adducts, ethoxylated mercaptan adducts, ethoxylated condensates with alkyl phenols, ethoxylated nitrogen-based hydrophobes, ethoxylated polyoxypropylenes, polymeric silicones, fluorinated surfactants, polymerizable surfactants, and mixtures thereof.

24. A method of stabilizing a polymerase enzyme in solution in the presence of a near-IR dye under conditions that normally inactivate the enzyme, the method comprising combining an effective amount of a nonionic surfactant with the enzyme and the dye, wherein the surfactant inhibits inactivation of the enzyme.

25. A method of conducting a thermal process, the method comprising:
providing a sample mixture comprising a biological material, an enzyme, an effective amount of a surfactant selected from the group of a nonionic surfactant, a zwitterionic surfactant, and a mixture thereof, and a dye at a first temperature; and
directly heating the sample mixture to a second temperature higher than the first temperature;
wherein the dye inactivates the enzyme in the absence of the surfactant and the surfactant inhibits the inactivation.

26. The method of claim 25 further comprising cooling the sample mixture and directly reheating the sample mixture in a thermal cycling process.

27. The method of claim 26 wherein the thermal cycling process comprises at least about 25 cycles.

28. The method of claim 27 wherein the first temperature is within a range of about 0° C. to about 50° C.

29. The method of claim 27 wherein the second temperature is within a range of about 50° C. to about 95° C.

30. The method of claim 27 wherein the thermal cycling process comprises heating between a temperature of about 50° C. and about 95° C.

31. A method of conducting a thermal cycling process, the method comprising:
providing a device comprising at least one process chamber that defines a volume for containing a sample mixture comprising a biological material, an enzyme, a dye, and a surfactant selected from the group of a nonionic surfactant, a zwitterionic surfactant, and a mixture thereof;
delivering electromagnetic energy to the process chamber to raise the temperature of the sample material in the process chamber, wherein the dye converts the electromagnetic energy into thermal energy;
wherein the surfactant inhibits interaction between the enzyme and the dye.

32. The method of claim 31 wherein the dye is a near-IR dye.

33. The method of claim 31 wherein the surfactant is a nonionic surfactant selected from the group of esters of fatty acids and polyhydric alcohols, fatty acid alkanolamides, ethoxylated fatty acids, ethoxylated aliphatic acids, ethoxylated fatty alcohols, ethoxylated aliphatic alcohols, ethoxylated sorbitol fatty acid esters, ethoxylated glycerides, ethoxylated block copolymers with EDTA, ethoxylated cyclic ether adducts, ethoxylated amide and imidazoline adducts, ethoxylated amine adducts, ethoxylated mercaptan adducts, ethoxylated condensates with alkyl phenols, ethoxylated nitrogen-based hydrophobes, ethoxylated polyoxypropylenes, polymeric silicones, fluorinated surfactants, polymerizable surfactants, and mixtures thereof.

34. The method of claim 31 wherein the sample mixture further comprises an antioxidant.

35. The method of claim 31 wherein the surfactant is present in an amount of at least about 0.5 wt-%.

36. The method of claim 35 wherein the surfactant is present in an amount of no greater than about 20 wt-%.

37. The method of claim 31 wherein the sample mixture further comprises a buffer.

38. The method of claim 31 wherein the sample mixture further comprises a dinucleotide triphosphate.

39. The method of claim 31 wherein the sample mixture further comprises a reference dye.

40. The method of claim 31 wherein the enzyme is a polymerase or a ligase.

41. A method of conducting a thermal cycling process comprising:
providing a device comprising at least one process chamber that defines a volume for containing a sample mixture comprising a biological material, a polymerase enzyme, a near-IR dye, a nonionic surfactant, and a dinucleotide triphosphate;
delivering electromagnetic energy to the process chamber to raise the temperature of the sample material in the process chamber, wherein the dye converts the electromagnetic energy into thermal energy;
wherein the surfactant inhibits interaction between the enzyme and the dye.

42. The method of claim 41 further comprising cooling the sample mixture and reheating the sample mixture in a thermal cycling process.

43. The method of claim 42 wherein the thermal cycling process comprises at least about 25 cycles.

44. The method of claim 42 wherein the thermal cycling process comprises heating between a temperature of about 50° C. and about 95° C.

45. A method of denaturing hydrogen-bonded molecules, the method comprising:
providing a sample mixture comprising hydrogen-bonded molecules, an enzyme, an effective amount of a surfactant selected from the group of a nonionic surfactant, a zwitterionic surfactant, and a mixture thereof, and a dye at a first temperature; and
directly heating the sample mixture to a second temperature higher than the first temperature effective to denature the hydrogen-bonded molecules;
wherein the dye inactivates the enzyme in the absence of the surfactant and the surfactant inhibits the inactivation.
